(19) United States
(12) Patent Application Publication
(10) Pub. No.: US 2003/0220594 A1
(43) Pub. Date: Nov. 27, 2003

(54) Torso orthosis apparatus and method

(75) Inventors: Kaia A. Halvorson, Seattle, WA (US); Paul D. LaBarre, Suquamish, WA (US)

Correspondence Address: Gerald L. Fellows, Michael Best & Friedrich LLP, 100 East Wisconsin Avenue, Milwaukee, WI, 53202-4108, US

(73) Assignee: United States Manufacturing Company, Inc., Poulsbo, WA (US)

(21) Appl. No.: 10/154,895

(22) Filed: May 24, 2002

(51) Int. Cl. A61F 5/00

(52) U.S. Cl. 602/19

(57) ABSTRACT

A torso orthosis including a chest support securable to a patient. The chest support includes a sternal plate securable in a substantially fixed position with respect to the patient's chest. The chest support also includes a sternal bar and a connection between the sternal plate and the sternal bar. The connection allows the sternal bar to move longitudinally with respect to the sternal plate so that the sternal plate remains in the substantially fixed position with respect to the patient's chest as the patient's torso flexes. The connection can also allow the sternal bar to pivot with respect to the sternal plate as the patient's torso flexes.

### FIELD OF THE INVENTION

This invention relates generally to torso orthoses, and more particularly to an apparatus and method for a torso orthosis including a chest support.

### BACKGROUND OF THE INVENTION

Torso orthoses are used to treat a variety of conditions associated with the thoracic lumbar skeletal structure (e.g., osteoporosis, back injuries, chest injuries, and spinal deformities) by applying pressure to selected positions along the patient's spine, abdomen, and torso. Typically, a torso orthosis includes at least one anterior support member secured to the patient's torso via one or more straps. The torso orthosis can also include a chest support member connected to the anterior support member that provides a posterior-directed pressure to the patient's chest.

One example of a torso orthosis is a thoracal lumbo-sacral orthosis (“TLSO”). A conventional TLSO typically includes an anterior torso shell connected to a posterior torso shell by straps that are wrapped around the patient's torso. A TLSO can also include a chest support member. When secured to the patient's body, the anterior torso shell provides a posterior-directed pressure to the patient's abdomen, the posterior torso shell provides anterior-directed pressure to the patient's spine and back, and the chest support member provides posterior-directed pressure to the patient's sternum and chest.

With a conventional TLSO, the chest support member is initially positioned in a desired location on the patient's chest and then rigidly connected to the anterior torso shell in order to generally resist forward movement of the patient's torso. When the patient is standing or lying down, the chest support member can be located at the desired position with respect to the patient's chest. However, when the patient sits down or bends over from a standing position or sits up from a lying down position, the anterior torso shell and the posterior torso shell remain secured to the patients' torso, but the chest support member moves upward from the desired position toward the patient's head. This movement of the chest support member upward from the desired position causes discomfort to the patient and can result in the chest support member being less effective. In addition, when the patient moves back to a standing or lying down position, the chest support member must be repositioned to the desired position. Thus, as the patient's torso flexes back and forth, the chest support member moves away from the desired position and must be repositioned often.

The chest support member of a conventional torso orthosis is also unable to remain in the desired position as the patient's torso twists from side to side. For example, if the patient's upper torso twists with respect to the patient's lower torso, a conventional chest support member will slide away from the desired position in a direction opposite to the direction that the patient's upper torso is twisting. Again, this movement of the chest support member away from the desired position causes discomfort to the patient, results in the chest support member being less effective, and requires the chest support member to be repositioned often.

In light of the problems and limitations described above, a need exists for a method and apparatus for a torso orthosis including a chest support that remains in a substantially fixed position with respect to a patient's chest when the patient's torso flexes. A need also exists for a chest support including a sternal plate, a sternal bar, and a connection between the sternal plate and the sternal bar that allows the sternal bar to move longitudinally with respect to the sternal plate. A need also exists for a chest support including a sternal plate, a sternal bar, and a connection between the sternal plate and the sternal bar that allows the sternal bar to pivot with respect to the sternal plate. A need further exists for a chest support for use with a number of different torso orthoses. Finally, a need exists for a chest support including a sternal plate, a sternal bar, a connection between the sternal plate and the sternal bar, and a lock that engages the sternal bar to adjust the distance the sternal bar can move with respect to the sternal plate.

### SUMMARY OF THE INVENTION

The present invention provides a chest support for use with a torso orthosis securable to a patient. The chest support includes a sternal plate and a sternal bar. The sternal plate is securable in a substantially fixed position with respect to the patient's chest. Some preferred embodiments of the chest support also include a connection between the sternal plate and the sternal bar that allows the sternal bar to move longitudinally so that the sternal plate remains in the substantially fixed position with respect to the patient's chest when the patient's torso flexes. The sternal bar preferably has an effective length which decreases and increases as the patient's torso bends forward and backward, respectively, with respect to the patient's pelvis. The sternal bar can increase in effective length due to a gravitational force acting on the sternal bar, or the sternal bar can be biased to increase in effective length. Some preferred embodiments include a connection that can also allow the sternal bar to pivot about one or more axis with respect to the sternal plate so that the sternal bar remains in the substantially fixed position with respect to the patient's chest when the patient's torso twists.

In some embodiments, the connection includes a housing having an exterior and an interior recess. The sternal plate can be connected to the exterior of the housing. The sternal bar can be slideably positioned within the interior recess so that the sternal bar slides with respect to the sternal plate. The sternal plate can also be pivotally coupled to the exterior of the housing so that the sternal bar pivots about one or more axis with respect to the sternal plate. For example, the sternal bar can pivot about either or both of a medial/lateral axis and a superior/inferior axis with respect to the sternal plate.

The sternal bar preferably includes a first or superior portion, a second or bend portion, and a third or inferior portion. The first portion can be connected to the sternal plate. Preferably, the first portion of the sternal bar is positionable substantially parallel to the patient's sternum. The second portion includes a bend which can serve to position the third portion of the sternal bar a suitable distance away from the patient chest. The third portion of the sternal bar preferably includes an extension that can be connected to a torso orthosis. For example, the third portion can include one or more holes through which one or more nuts, screws, bolts, or other suitable fasteners are positioned in order to secure the sternal bar to the torso orthosis.

In some embodiments, the chest support includes a lock that engages the sternal bar in order to adjust the distance that the sternal bar moves longitudinally with respect to the sternal plate. The lock can include a first or engagement position in which the sternal bar cannot move longitudinally with respect to the sternal plate and a second or non-engagement position in which the sternal bar can move longitudinally with respect to the sternal plate. The sternal bar can also include a plurality of indentations and the lock can include a set screw selectively positionable within each one of the plurality of indentations.

According to some methods of the invention, a patient's torso is supported by positioning a sternal plate into contact with the patient's chest in a substantially fixed position. The method includes connecting a sternal bar to the sternal plate and moving the sternal bar longitudinally as the patient's torso flexes so that the sternal plate remains in the substantially fixed position with respect to the patient's chest. The method can also include biasing the sternal bar to increase in effective length as the patient's torso bends backward with respect to the patient's pelvis. The method also preferably includes pivoting the sternal bar about one or more axis with respect to the sternal plate as the patient's torso twists. The method can also include adjusting the distance that the sternal bar moves longitudinally with respect to the sternal plate. The method can still further include locking the sternal bar so that the sternal bar cannot move longitudinally with respect to the sternal plate.

Further objects and advantages of the present invention, together with the organization and manner of operation thereof, will become apparent from the following detailed description of the invention when taken in conjunction with the accompanying drawings, wherein like elements have like numerals throughout the drawings.

### BRIEF DESCRIPTION OF THE DRAWINGS

The present invention is further described with reference to the accompanying drawings which show preferred embodiments of the present invention. However, it should be noted that the invention as disclosed in the accompanying drawings is illustrated by way of example only. The various elements and combinations of elements described below and illustrated in the drawings can be arranged and organized differently to result in embodiments which are still within the spirit and scope of the present invention.

In the drawings, wherein like reference numerals indicate like parts:

FIG. 1 is a perspective view of a chest support according to a preferred embodiment of the invention;

FIG. 2 is a front elevational view of the chest support of FIG. 1;

FIG. 3 is a back elevational view of the chest support of FIG. 1;

FIG. 4 is a side sectional view of the chest support taken along line 4-4 of FIG. 2;

FIG. 5 is an exploded front perspective view of the chest support of FIG. 1;

FIG. 6 is an exploded back perspective view of the chest support of FIG. 1, showing a number of the elements illustrated in FIG. 5 removed for clarity;

FIG. 7 is a partial side view of a patient lying in a prone position and wearing a thoracal lumbo-sacral orthosis including a chest support according to a preferred embodiment of the invention;

FIG. 8 is a partial side view of a patient in a seated position and wearing a thoracal lumbo-sacral orthosis including a chest support according to a preferred embodiment of the invention;

FIG. 9 is a perspective view of a patient wearing a thoracal lumbo-sacral orthosis including a chest support according to a preferred embodiment of the invention;

FIG. 10 is a perspective view of a patient wearing a lumbo-sacral orthosis including a chest support according to a preferred embodiment of the invention;

FIG. 11 is a perspective view of a patient wearing a cervical thoracal lumbo-sacral orthosis including a chest support according to a preferred embodiment of the invention;

FIGS. 12A and 12B are front and back perspective views, respectively, of a patient wearing a cruciform anterior spinal hyperextension orthosis including a chest support according to a preferred embodiment of the invention;

FIGS. 13A and 13B are front and back perspective views, respectively, of a patient wearing a Danforth hyperextension orthosis including a chest support according to a preferred embodiment of the invention; and

FIG. 14 is a perspective view of a patient wearing a cruciform anterior spinal hyperextension orthosis including another embodiment of a chest support according to a preferred embodiment of the invention.

### DETAILED DESCRIPTION OF THE PREFERRED EMBODIMENTS

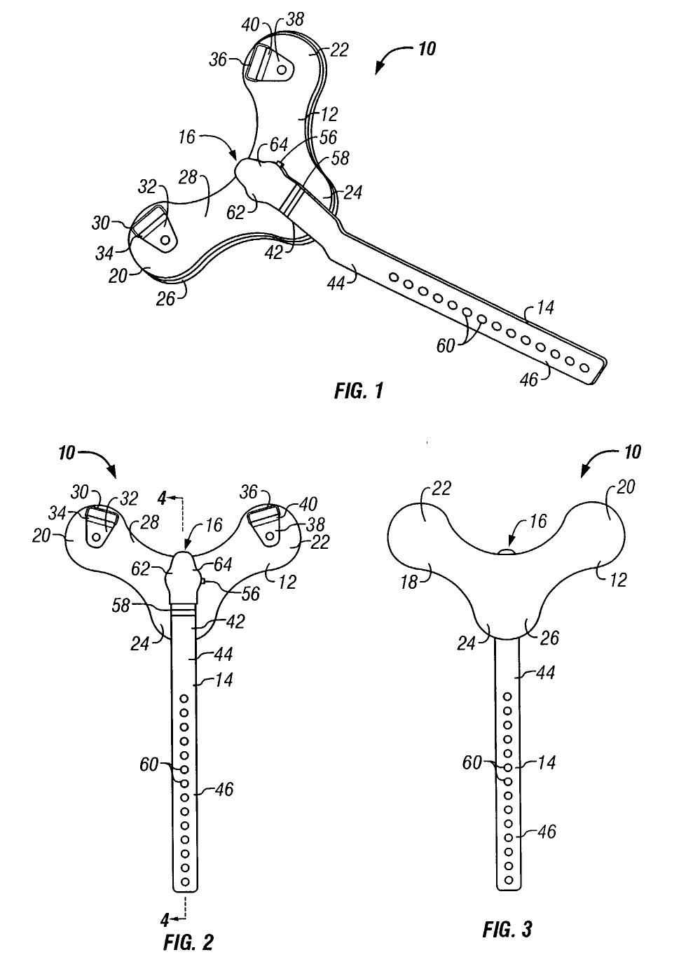

FIGS. 1-6 illustrate a chest support 10 embodying the invention. The chest support 10 includes a sternal plate 12, a sternal bar 14, and a connection 16 between the sternal plate 12 and the sternal bar 14.

As shown in FIG. 3, the sternal plate 12 includes a contact surface 18 that can be positioned into contact with a patient's chest. The contact surface 18 is preferably contoured slightly to conform to the patient's chest. As shown in FIGS. 1-3, the sternal plate 12 preferably includes a first lateral lobe 20, a second lateral lobe 22, and a medial lobe 24. The first lateral lobe 20 and the second lateral lobe 22 are preferably integral with the medial lobe 24. However, each of the lobes 20, 22, and 24 can be separate components coupled together in any suitable manner (e.g., by various brace members, bars, extensions, etc.). The first lateral lobe 20 and the second lateral lobe 22 are preferably shaped and positioned to contact the right and left sides, respectively, of the patient's chest. The medial lobe 24 is preferably shaped and positioned to contact the patient's chest near the patient's sternum.

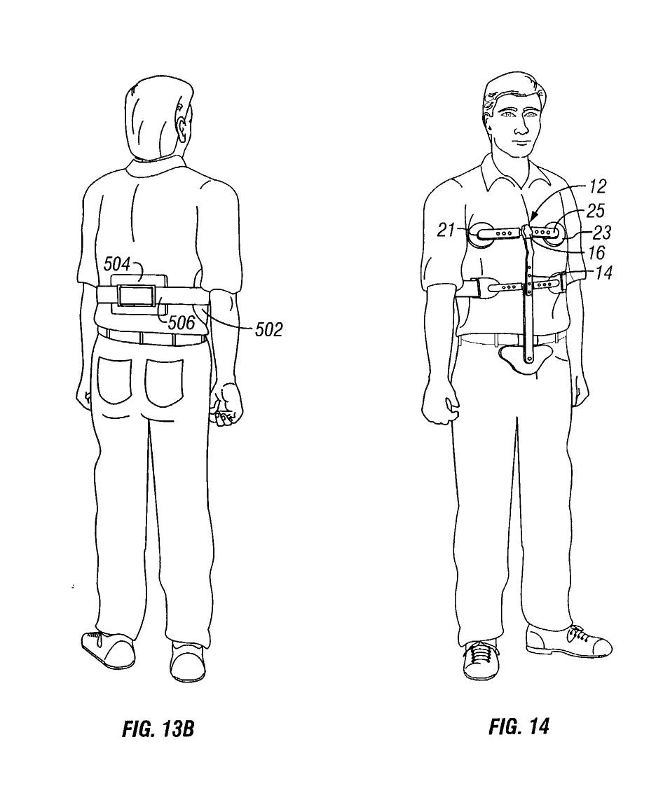

Although shown and described as having lobes 20, 22, and 24, the sternal plate 12 can include one or more other pad members having any suitable shape, such as a triangular shape, a rectangular shape, a circular shape, an oval shape, or any other suitable shape or combination of shapes. One of ordinary skill in the art will appreciate that the sternal plate 12 can also include separate pad members which can be placed at a variety of positions on the patient's chest. For example, as shown in FIG. 14, the sternal plate 12 can include a first lateral lobe 21 and a separate, second lateral lobe 23 coupled together by a horizontally-positioned bar 25, without having a medial lobe.

When the sternal plate 12 is connected to a torso orthosis being worn by a patient (as will be described in detail below), the sternal plate 12 provides a force in a posterior direction against the patient's chest in order to resist forward movement of the patient's torso. Preferably, the sternal plate 12 is held in a substantially fixed position on the patient's chest by a frictional force between the contact surface 18 of the sternal plate 12 and the patient's chest. In addition to the frictional force, the sternal plate 12 can be held in the substantially fixed position in a number of different manners, such as by attaching straps to the sternal plate 12 and wrapping the straps over the patient's shoulders or around the patient's back, as will be described in detail below.

As shown in FIG. 1, a lining 26 is preferably attached to the contact surface 18 of the sternal plate 12. The lining 26 is preferably a soft, thermo-absorbent material that provides a comfortable contact surface and conducts heat away from the patient's body. The lining 26 also provides an additional frictional force between the sternal plate 12 and the patient's chest. The lining 26 is preferably coupled to the contact surface 18 by hook-and-loop fasteners, so that the lining 26 can be easily removed and replaced. However, the lining 26 can be connected to the contact surface 18 in any suitable manner, such as with adhesives, elastic bands, snap-fit connections, buckles, clips, rivets, press-fit connections, by integrating the lining 26 with the contact surface 18, etc. Also, the lining 26 can include an elastic band connected to its perimeter so that the elastic band can be stretched and wrapped around the perimeter of the sternal plate 12. One preferred type of material for the lining 26 is a styrene butadiene foam preferably approximately 1 mm to 10 mm thick (e.g., THERMASORB® by Frisby Technologies, Inc.). The lining 26 can also be constructed of one or more other suitable materials (whether capable of directing heat away from the patient's body or not), such as closed-cell foams, soft plastics, or fabrics (e.g., cotton and cotton blends).

As shown in FIGS. 1 and 2, the sternal plate 12 also includes a non-contact surface 28 (on a side opposite to the contact surface 18). In some embodiments, a first ring 30 and a second ring 36 are used to connect straps that extend over the patient's shoulders to the sternal plate 12. The first ring 30 and the second ring 36 can be rectangular-shaped, circular-shaped, oval-shaped, or any other shape suitable for accommodating the shape of the particular shoulder straps. The first ring 30 is preferably pivotally coupled to the non-contact surface 28 on the first lateral lobe 20 by a first attachment plate 32. The first attachment plate 32 preferably includes a cylindrical opening 34 through which a portion of the first ring 30 is pivotally positioned. The second ring 36 is preferably pivotally coupled to the non-contact surface 28 on the second lateral lobe 22 by a second attachment plate 38. The second attachment plate 38 also preferably includes a cylindrical opening 40 through which a portion of the second ring 36 is pivotally positioned. However, one or more rings can be attached to the sternal plate 12 in any suitable manner. One or more rings can also be attached in other suitable positions with respect to the sternal plate 12 (e.g., to the lateral edges of the sternal plate 12 for a strap around the patient's back). Rather than rings, other releasable or non-releasable fasteners or mechanisms can be used to attach straps to the sternal plate 12 (e.g., adhesives, laces, hooks and loops, snap-fit connections, buckles, press-fit connections, bolts, pins, etc.). Moreover, the rings 30, 36 can be omitted from the sternal plate 12 if additional straps will not be used with a particular torso orthosis.

The sternal plate 12 is preferably constructed of aluminum. However, the sternal plate 12 can be constructed of one or more other suitable materials, such as composites, plastics, titanium, fiberglass, steel, other metals and metal alloys, polymers, etc.

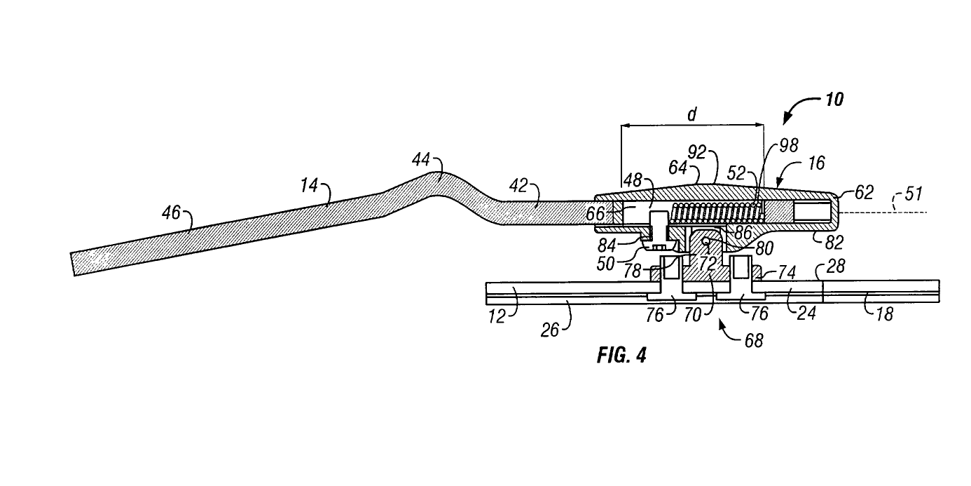

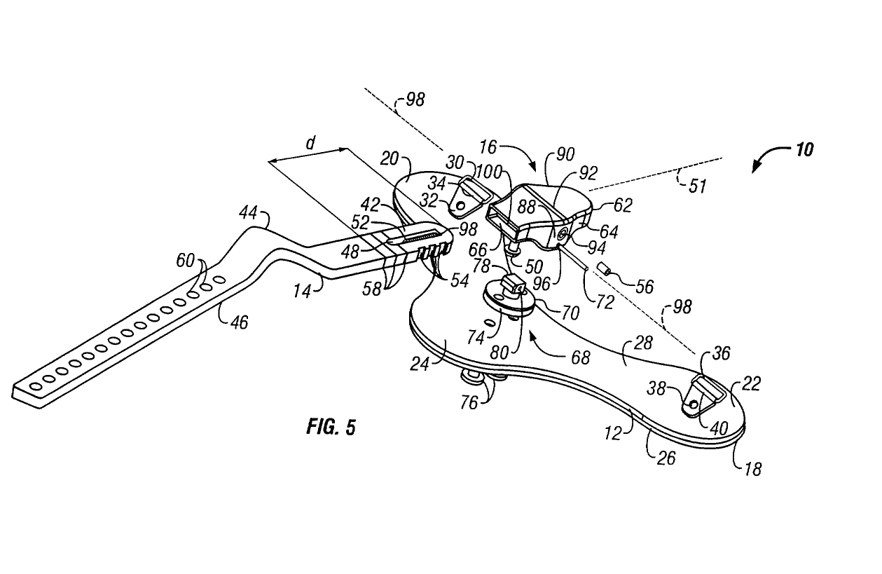

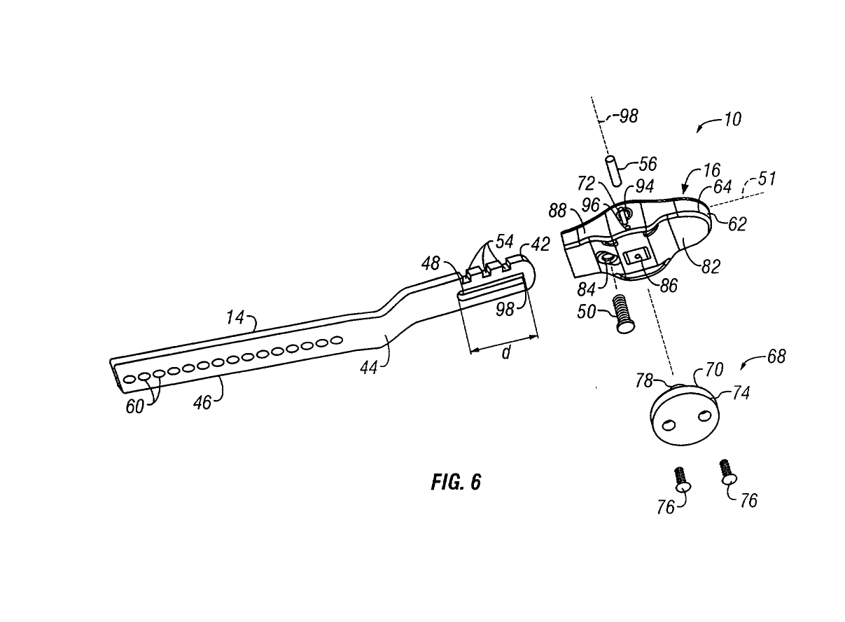

As best shown in FIGS. 1 and 5, the sternal bar 14 preferably includes a superior portion 42, a bend portion 44, and an inferior portion 46. The superior portion 42 is preferably connected to the sternal plate 12 by the connection 16. The superior portion 42 is preferably dimensioned and positionable so that the superior portion 42 lies parallel to the patient's sternum when the chest support 10 is attached to the patient's chest. As shown in FIGS. 4-6, the superior portion 42 preferably includes a slot 48 which receives a retention screw 50 or another suitable fastener, extension, or member. The slot 48 defines a distance d that the sternal bar 14 can move with respect to the sternal plate 12. Specifically, the sternal bar 14 moves along an axis that lies perpendicular to the traverse or horizontal plane (which divides the patient's body into superior and inferior portions), i.e., a superior/inferior axis 51. A spring 52 is preferably positioned within the length of the slot 48 in order to bias the sternal bar 14 downward, as will be described in more detail below. As shown in FIGS. 5 and 6, the superior portion 42 of the sternal bar 14 also preferably includes one or more indentations or lock-out detents 54. The lock-out detents 54 preferably receive a set screw 56 or another suitable locking mechanism in order to prevent the sternal bar 14 from moving longitudinally. In addition, as shown in FIGS. 1, 2 and 5, the superior portion 42 of the sternal bar 14 preferably includes scribe marks 58 corresponding to the positions of the lock-out detents 54. The scribe marks 58 are preferably used by the patient or a clinician to align the connection 16 with a particular lock-out detent 54.

The bend portion 44 of the sternal bar 14 preferably includes a bend which serves to position the inferior portion 46 more distally from the patient's chest than the superior portion 42. In some embodiments, as best shown in FIG. 4, the bend is sigmoid-shaped (“S”-shaped). However, the bend can also be a right-angle bend, a convex bend, a concave bend, a sloped bend, or any other desired shape.

As shown in FIGS. 1-3 and 5-6, the inferior portion 46 of the sternal bar 14 preferably includes an extension having several holes 60. The holes 60 preferably receive T-nuts or other suitable fasteners in order to couple the chest support 10 to a torso orthosis, as will be described in more detail below. If necessary, a clinician can cut off an extraneous part of the inferior portion 46 of the sternal bar 14 once the torso orthosis has been fitted for the particular patient.

The sternal bar 14 preferably has a rectangular cross-sectional shape (as best shown in FIG. 5). However, the sternal bar 14 can have any suitable cross-sectional shape or any suitable combinations of cross-sectional shapes (rather than or in addition to the rectangular shape shown and described with respect to the drawings). The superior portion 42, the bend portion 44, and the inferior portion 46 of the sternal bar 14 can have the same cross-sectional shape or different cross-sectional shapes. Some examples of suitable cross-sectional shapes are an X-shape, a T-shape, a square shape, a circular shape, an oval shape, or any combination thereof. Accordingly, the superior portion 42 can have a circular cross-sectional shape, while the bend portion 44 and the inferior portion 46 have rectangular cross-sectional shapes. Moreover, the sternal bar 14 can be comprised of two or more bars, rods, extensions, etc. For example, the sternal bar 14 can include two parallel rods each having a circular cross-sectional shape.

The sternal bar 14 is preferably constructed of aluminum, so that a clinician can bend the sternal bar 14 to conform to the patient's chest, if necessary. However, the sternal bar 14 can be constructed of one or more other suitable materials, such as composites, plastics, titanium, fiberglass, steel, other metals and metal alloys, polymers, etc.

The connection 16 is preferably positioned between the non-contact surface 28 of the sternal plate 12 and the superior portion 42 of the sternal bar 14. In one highly preferred embodiment (as best shown in FIGS. 4-6), the connection 16 includes a housing 62 having an exterior 64 and an interior recess 66. Preferably, the housing 62 is integrally constructed of a molded polymer. The material for the housing 62 also preferably has enhanced lubricity. For example, the housing 62 can be constructed of an acetal polymer (e.g., DELRIN® by E. I. duPont de Numours and Company). The housing 62 can also be constructed of one or more other suitable materials (whether integrally molded or constructed of assembled components), such as ERTALYTE® (Quadrant DSM Polymer Corporation), Noryl® (General Electric Company), UHMW Polyethylene or one of its commercial versions Lenite® (Westlake Plastics Company) or Tivar 1000® (Poly Hi Solidue, Inc.), hardened polymers, steel and other metals, metal alloys, plastics, nylon, aluminum, fiberglass, carbon-fiber composite, etc.

As shown in FIGS. 4 and 6, the exterior 64 of the housing 62 of the connection 16 includes a proximal surface 82. The proximal surface 82 includes a retention screw hole 84 and a rocker stud receiving hole 86. The housing 62 of the connection 16 also includes a first lateral surface 88 and a second lateral surface 90 (as shown in FIG. 5). The housing 62 also preferably includes an increased-width portion 92 between the first lateral surface 88 and the second lateral surface 90. Within the increased-width portion 92 on the first lateral surface 88, the housing 62 preferably includes a set screw receiving hole 94 and a pivot pin receiving aperture 96.

As shown in FIGS. 4-6, in order to couple the sternal bar 14 to the connection 16, the superior portion 42 of the sternal bar 14 is positioned within the interior recess 66 of the housing 62. The retention screw 50 is positioned into the retention screw receiving hole 84. The retention screw 50 extends into the slot 48 of the superior portion 42 of the sternal bar 14. In this manner, the sternal bar 14 is slideably coupled to the housing 62. The retention screw 50 can be any shaft, extension, or member that is able to move relatively freely along the superior/inferior axis 51 within the slot 48, preferably with little lateral movement within the slot 48. Typically, the diameter of the retention screw 50 is slightly less than the width of the slot 48 to permit relatively free movement along the superior/inferior axis 51 without significant lateral movement within the slot 48. One of ordinary skill in the art will recognize that a variety of bolts, screws, rivets, pins, extensions, etc. are suitable for use as the retention screw 50. In some embodiments, the spring 52 is also positioned within the length of the slot 48 of the superior portion 42 of the sternal bar 14. The spring 52 provides a biasing force between the retention screw 50 and a superior surface 98 of the slot 48. Preferably, the spring 52 biases the sternal bar 14 downward (i.e., toward the patient's pelvis) along the superior/inferior axis 51, so that the sternal bar 14 increases in length as the patient's torso flexes backward with respect to the patient's pelvis. Rather than or in addition to the spring 52, other suitable biasing mechanisms can be used to bias the sternal bar 14 downward. For example, a counter-weight can be coupled to the sternal bar 14 or the connection 16 in a selected position to bias the sternal bar 14. Also, one or materials having increased or decreased coefficients of friction can be inserted into the slot 48. In other embodiments, the spring 52 is omitted and only gravitational forces bias the sternal bar 14 downward along the superior/inferior axis 51.

As shown in FIGS. 4-6, the connection 16 can also include a hinge 68, preferably comprised of a rocker stud 70 and a pivot pin 72. The rocker stud 70 includes a base 74 which is preferably rigidly coupled to the medial lobe 24 (as shown in FIG. 5) of the sternal plate 12 via attachment rivets 76 or any other suitable fasteners. The rocker stud 70 also includes a boss 78 with a cylindrical aperture 80 adapted to receive the pivot pin 72. The pivot pin 72 is positioned into the pivot pin receiving aperture 96 of the housing 62 and through the cylindrical aperture 80 in the boss 78 of the rocker stud 70. In this manner, the sternal plate 12 is pivotally coupled to the connection 16 so that the sternal plate 12 remains in a substantially fixed position when the patient's torso twists, flexes, and/or bends in any manner.

Although the hinge 68 is shown and described as including the rocker stud 70 and the pivot pin 72, the hinge 68 can be any mechanism capable of allowing the sternal bar 14 to pivot in one or more directions with respect to the sternal plate 12. For example, the hinge 68 can include one or more plates connected to the sternal plate 12 and the sternal bar 14 that rotate about a shaft. The hinge 68 can also be a living hinge constructed of one or more pieces of flexible material, such as a relatively flexible piece of plastic including a scored portion or other hinges known in the art. Moreover, rather than a hinge 68, the sternal plate 12 can be coupled to the sternal bar 14 via a ball and socket joint or any other suitable joint mechanism.

In one highly preferred embodiment, the hinge 68 (e.g., the rocker stud 70 and the pivot pin 72) is positioned so that the connection 16 and the sternal bar 14 pivot about an axis that lies in the medial/lateral plane (also referred to as the frontal or coronal plane) with respect to the patient's body, i.e., a medial/lateral axis 98 (as shown in FIGS. 5 and 6). However, in some embodiments, the hinge 68 is positioned so that the sternal bar 14 pivots about the superior/inferior axis 51. In still other embodiments, one or more pivoting members can be positioned so that the sternal bar 14 pivots about the superior/inferior axis 51, the medial/lateral axis 98, or any combination thereof. For example, rather than the rocker stud 70, a ball and socket joint can be used to rotatably couple the sternal plate 12 to the connection 16 and the sternal bar 14. If a ball and socket joint is used, bumpers or other movement-limiting members are preferably provided in order to somewhat limit the motion of the ball and socket joint. For example, bumpers can be coupled to the sternal plate 12 in order to limit the ball and socket joint to one or more particular degrees of motion.

Although the sternal bar 14 preferably moves longitudinally and pivotally with respect to the sternal plate 12, the sternal bar 14 can optionally be fixed with respect to the sternal plate 12. To prevent longitudinal movement of the sternal bar 14, a user (i.e., a patient or a clinician) can preferably adjust the set screw 56 located on the first lateral side 88 of housing 62 (although the set screw 56 can be located anywhere on the housing 62 that corresponds to the lock-out detents 54 or any other suitable lock mechanism) in order to fix the position of the sternal bar 14 with respect to the housing 62. The user moves the sternal bar 14 into or out of the interior recess 66 of the housing 62. As shown in FIG. 5, the user preferably aligns the scribe marks 58 with an interior edge 100 of the housing 62 in order to set the desired length for the sternal bar 14 and to align the set screw receiving hole 94 with one of the lock-out detents 54. Once the desired length for the sternal bar 14 is determined, the user tightens the set screw 56 into the set screw receiving hole 94 so that the set screw 56 is positioned within one of the lock-out detents 54. In this manner, the set screw 56 engages the edges of the particular lock-out detent 54 to prevent the sternal bar 14 from moving longitudinally with respect to the sternal plate 12. Longitudinal movement of the sternal bar 14 can also be optionally prevented in various other manners (e.g., by inserting additional limiting members into the housing 62 or by attaching a collar around the perimeter of the sternal bar 14).

Rather than preventing longitudinal movement, in some embodiments, the user can adjust the distance d that the sternal bar 14 can move with respect to the sternal plate 12. For example, rather than being positioned into one of the lock-out detents 54, the set screw 56 (or any other suitable shaft, extension, or member) could extend into the slot 48 in order to reduce the effective length of the slot 48. With the effective length of the slot 48 reduced, the retention screw 50 travels a shorter distance along the superior/inferior axis 51. In this manner, the effective length of the slot 48 can be varied to allow from full travel to zero travel for the retention screw 50. The distance d that the sternal bar can move longitudinally can be varied in other suitable manners (e.g., by inserting additional limiting members into the housing 62 or by attaching a collar around the perimeter of the sternal bar 14).

Although shown and described as including the housing 62 and the hinge 68, the connection 16 can be comprised of any number of other suitable connectors, linkages, and/or hinges. For example, the connection 16 can be comprised of one or more links rotatably coupled between the sternal plate 12 and the sternal bar 14. The links can add to the effective length of the sternal bar 14 when the patient's torso flexes backward, but collapse to reduce the effective length of the sternal bar 14 when the patient's torso flexes forward. As discussed above, the sternal bar 14 can have any suitable cross-sectional shape (e.g., an X-shape, a T-shape, a square shape, a circular shape, an oval shape, or any combination thereof) and can be comprised of two or more bars, rods, extensions, etc. The connection 16 can then be adapted to connect the sternal plate 12 to a particular type of sternal bar 14. For example, the sternal bar 14 can be comprised of a single rod having a circular cross-section. Accordingly, the connection 16 can include a sleeve or other suitable housing having a circular aperture adapted to slideably and pivotally connect to the single circular rod of the sternal bar 14. The sternal bar 14 can then slide longitudinally and pivot laterally with respect to the sternal plate 12. As another example, the sternal bar 14 can be comprised of two parallel rods. The connection 16 can then include an extension connected to the sternal plate 12 and slideably positioned between the two parallel rods of the sternal bar 14. The extension can slide longitudinally and pivot laterally between the two parallel rods.

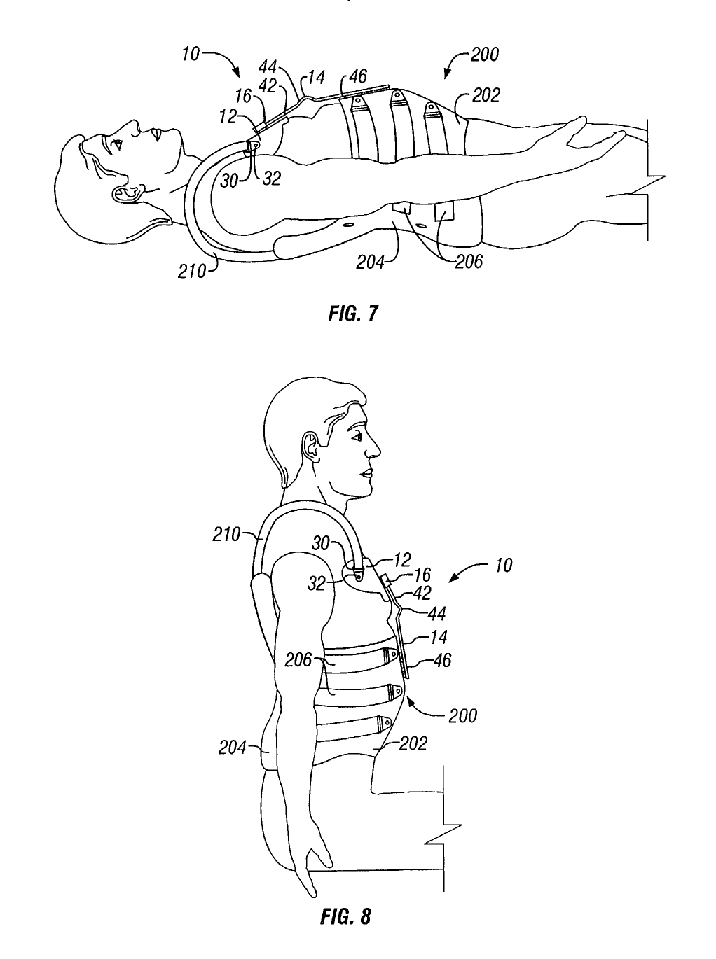

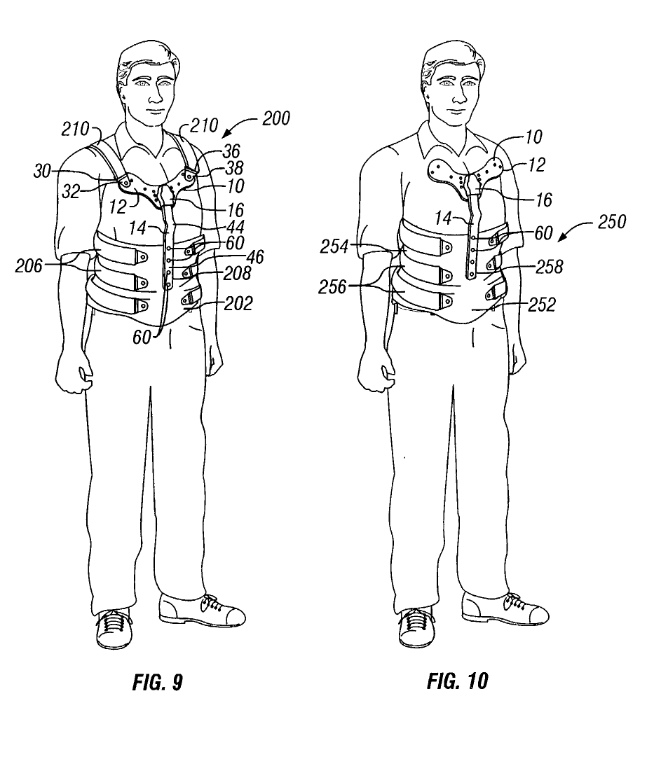

The chest support 10 shown and described with respect to FIGS. 1-6 can be used in conjunction with several different types of orthotic braces or torso orthoses. The term “orthotic brace” as used herein and in the appended claims refers to any device suitable for supporting a portion of a patient's body. The term “torso orthosis” as used herein and in the appended claims refers to any device suitable for supporting or bracing a patient's torso. As shown in FIGS. 7-9, the chest support 10 is used in conjunction with a thoracal lumbo-sacral orthosis (“TLSO”) 200. The TLSO 200 includes an anterior torso shell 202 conformed to a patient's abdomen and a posterior torso shell 204 conformed to a patient's thoracic lumbar column. The anterior torso shell 202 is preferably attached to the posterior torso shell 204 by one or more pairs of straps 206 that can be wrapped and adjusted around the patient's torso and waist. The straps 206 are preferably adjusted in order for the anterior torso shell 202 to provide posterior-directed pressure to the patient's abdomen and for the posterior torso shell 204 to provide anterior-directed pressure to the patient's spine.

The chest support 10 is attached to the anterior torso shell 202 of the TLSO 200 in order to provide a posterior-directed pressure at a substantially fixed position with respect to the patient's chest. Preferably, the inferior portion 46 of the sternal bar 14 is connected to the anterior torso shell 202 so that the bend portion 44 of the sternal bar 14 extends above the anterior torso shell 202. Preferably, T-nuts or other suitable fasteners are positioned and secured through one or more of the holes 60 in the inferior portion 46 of the sternal bar 14 and through holes 208 in the anterior torso shell 202. However, the chest support 10 can be coupled to the TLSO 200 in any suitable manner (e.g., with bolts, screws, buckles, clips, mating pins and apertures, rivets, threaded connections, snap-fit connections, press-fit connections, etc.) or the chest support 10 can be integral with the TLSO 200. For example, the sternal bar 14 can be integrally constructed of the same material as the anterior torso shell 202 and extend upward from the anterior torso shell 202 toward the patient's chest.

The TLSO 200 also preferably includes a pair of shoulder straps 210 connected to the posterior torso shell 204 and coupleable to the sternal plate 12 in any suitable manner. For example, the shoulder straps 210 can be coupled to the sternal plate 12 via the first ring 30, the first attachment plate 32, the second ring 36, and the second attachment plate 38. The interior surfaces of the anterior torso shell 202 and the posterior torso shell 204 are preferably lined with a lining (e.g., a lining similar to the lining 26 attached to the contact surface 18 of the sternal plate 12 or any other suitable lining).

As shown in FIG. 7, when a patient wearing the TLSO 200 is lying in a prone position (or in a standing position), the sternal plate 12 contacts the patient's chest in a substantially fixed position and the sternal bar 14 is substantially extended (i.e., the effective length of the sternal bar 14 is increased). As shown in FIG. 8, as the patient's torso flexes toward the patient's pelvis when the patient moves to a seated position (or bends over from a standing position), the sternal plate 12 remains in the substantially fixed position. However, the sternal bar 14 moves longitudinally along the superior/inferior axis 51. As a result, the sternal bar 14 decreases in effective length.

In the embodiment shown in FIGS. 1-6, the superior portion 42 of the sternal bar 14 moves farther into the interior recess 66 of the housing 62 in order to decrease in effective length. In addition to the longitudinal movement of the sternal bar 14 along the superior/inferior axis 51, the sternal bar 14 also preferably pivots about the medial/lateral axis 94 with respect to the sternal plate 12. When the patient lies down again (or stands upright from a bent over position or stands up from a seated position), the patient's torso flexes away from the patient's pelvis and the sternal bar 14 moves out of the interior recess 66 of the housing 62 in order to increase in effective length. The sternal bar 14 also preferably pivots about the medial/lateral axis 94 when the patient lies down or stands upright. In this manner, the sternal plate 12 remains in the substantially fixed position as the patient's torso flexes forward or backward with respect to the patient's pelvis.

In general, when the patient's body elongates in any manner (e.g., by twisting, extending, or stretching), the sternal plate 12 remains in the substantially fixed position and the sternal bar 14 increases in effective length. Conversely, when the patient's body shortens in any manner (e.g., by bending, flexing, contracting, or compressing), the sternal plate 12 remains in the substantially fixed position and the sternal bar 14 decreases in effective length.

In some embodiments, the sternal bar 14 also pivots about the superior/inferior axis 51 with respect to the sternal plate 12. Thus, when the patient's torso twists about the superior/inferior axis 51, the sternal bar 14 pivots about the superior/inferior axis 51. In some embodiments, the sternal bar 14 can pivot relatively freely about a ball and socket joint with respect to the sternal plate 12 (i.e., when a ball and socket joint is used). Accordingly, when the patient's torso twists, flexes, or bends, the sternal bar 14 pivots relatively freely about 360 degrees of motion, unless otherwise limited.

As shown in FIG. 10, the chest support 10 can be used in conjunction with a lumbo-sacral orthosis (“LSO”) 250. The LSO 250 is substantially similar to the TLSO 200, and includes an anterior torso shell 252, a posterior torso shell 254, pairs of straps 256, and holes 258. However, the LSO 250 does not include shoulder straps. Accordingly, the chest support 10 does not need to include the rings 30, 36 attached to the sternal plate 12.

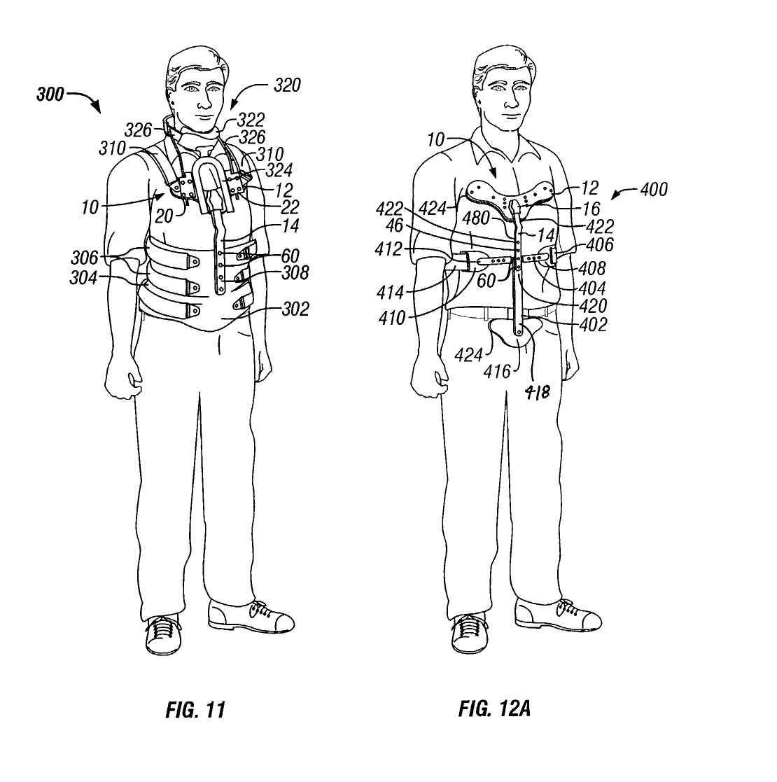

As shown in FIG. 11, the chest support 10 can be used in conjunction with a cervical, thoracal lumbo-sacral orthosis (“CTLSO”) 300. The CTLSO 300 is substantially similar to the TLSO 200, and includes an anterior torso shell 302, a posterior torso shell 304, pairs of straps 306, holes 308, and shoulder straps 310. However, in addition to the components included with the TLSO 200, the CTLSO 300 includes a cervical attachment 320. The cervical attachment 320 can be any device designed to support the patient's neck and cervical vertebrae with respect to the patient's torso. The cervical attachment 320 preferably includes a cervical collar 322, a cervical extension 324, and support rods 326. Preferably, the cervical collar 322 is adjustable to conform to the patient's neck size. The cervical extension 324 is preferably rigidly connected between the sternal plate 12 and the cervical collar 322. The cervical extension 324 can be a separate component from the sternal bar 12, or the cervical extension 324 can be integrally connected to the sternal plate 12. The support rods 326 preferably provide additional lateral support for the cervical collar 322. The support rods 326 are preferably rigidly connected between the cervical collar 322 and the first lateral lobe 20 and the second lateral lobe 22 of the sternal plate 12. In general, the cervical attachment 320 remains substantially fixed with respect to the sternal plate 12 as the sternal bar 14 moves longitudinally and/or pivots.

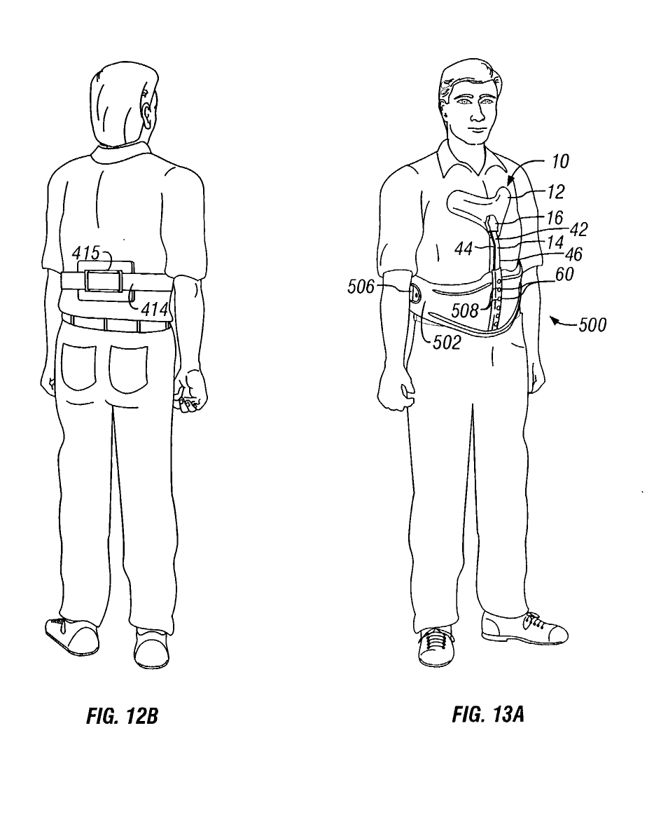

As shown in FIGS. 12A and 12B, the chest support 10 can be used in conjunction with a hyperextension orthosis or a torso anterior brace, such as a cruciform anterior spinal hyperextension orthosis 400. The hyperextension orthosis 400 includes a longitudinal brace member 402 connected to a lateral brace member 404 positioned perpendicular to the longitudinal brace member 402. Preferably, the longitudinal brace member 402 and the lateral brace member 404 are adjustable with respect to one another in any suitable manner. For example, nuts, screws, bolts, or any other suitable releasable or non-releasable fasteners can be positioned and secured through holes or other apertures in one or both of the longitudinal brace member 402 and the lateral brace member 404.

The hyperextension orthosis 400 also includes a left abdominal pad 406 connected to a left end 408 of the lateral brace member 404 and a right abdominal pad 410 connected to a right end 412 of the lateral brace member 404. Preferably, the left abdominal pad 406 and the right abdominal pad 410 are adjustable with respect to the lateral brace member 404. For example, nuts, screws, bolts, or any other suitable releasable or non-releasable fasteners can be positioned and secured through holes or other apertures on one or both of the abdominal pads 406, 410 and the lateral brace member 404. Also, the abdominal pads 406, 410 can include slots within which one or more followers (e.g., nuts, screws, bolts, extensions, or other suitable members) connected to the lateral brace member 404 can slide. The left abdominal pad 406 and the right abdominal pad 410 are preferably connected to one another by one or more straps 414 positioned and secured across the patient's back (as shown in FIG. 12B). Preferably, the straps 414 are adjustable and are also used to secure the entire hyperextension orthosis 400 to the patient. The ends of the straps 414 are preferably fastened to one another with hook and loop fasteners for touch-close fastening (i.e., the patient only needs one free hand to fasten the straps). However, the ends of the straps 414 can also be fastened to one another by hooks and loops, snaps, buckles, laces, clips, by being tied to one another, etc.

As shown in FIG. 12B, a posterior pad 415 is also preferably connected to the straps 414. The posterior pad 415 is positioned on the patient's back in order to provide an anterior-directed pressure to the patient's spine. Preferably, the posterior pad 415 is adjustable with respect to the straps 414 in order to properly center the posterior pad 415 with respect to the patient's spine. For example, the posterior pad 415 can include slots that slideably receive the straps 414. Also for example, the posterior pad 415 can also be connected to the straps 414 with snap-fit or press-fit connections at various locations along the length of the straps 414.

The hyperextension orthosis 400 also includes a pelvic pad 416 connected to an inferior end 418 of the longitudinal brace member 402. The pelvic pad 416 provides a posterior-directed pressure to the patient's pelvic region. Preferably, the pelvic pad 416 is adjustable with respect to the longitudinal brace member 402 in order to properly position the pelvic pad 416 with respect to the patient's pelvis. For example, nuts, screws, bolts, or any other suitable releasable or non-releasable fasteners can be positioned and secured through holes or other apertures on one or both of the pelvic pad 416 and the longitudinal brace member 402. Also, the pelvic pad 416 can include slots within which one or more followers (e.g., nuts, screws, bolts, extensions, or other suitable members) connected to the longitudinal brace member 402 can slide.

The chest support 10 is attached to the longitudinal brace member 402 of the hyperextension orthosis 400 in order to provide a posterior-directed pressure at a substantially fixed position with respect to the patient's chest. Preferably, the inferior portion 46 of the sternal bar 14 is connected to a superior end 420 of the longitudinal brace member 402. Preferably, T-nuts or other suitable fasteners are positioned and secured through one or more of the holes 60 in the inferior portion 46 of the sternal bar 14 and through holes 422 in the longitudinal brace member 402. However, the chest support 10 can be connected to the hyperextension orthosis 400 in any suitable manner (e.g., with bolts, screws, buckles, clips, mating pins and apertures, rivets, threaded connections, snap-fit connections, press-fit connections, and the like) or the chest support 10 can be integral with the longitudinal brace member 402 of the hyperextension orthosis 400. For example, the sternal bar 14 can be integrally constructed of the same material as the longitudinal brace member 402 and can extend upward from the longitudinal brace member 402 toward the patient's chest.

Preferably, the left abdominal pad 406, the right abdominal pad 410, the posterior pad 415, the pelvic pad 416, and the sternal pad 12 are each constructed of a relatively rigid material at least partially covered with a lining 424. For example, the pads can be constructed of one or more materials, such as aluminum, composites, plastics, titanium, fiberglass, steel, other metals and metal alloys, polymers, etc. Preferably, the lining 424 is a thermo-absorbent material, as described above with respect to the lining 26 attached to the contact surface 18 of the sternal plate 12. However, the lining 424 can be any material suitable for providing a contact surface between the respective pads and the patient's torso (even without being capable of directing heat away from the patient's body). The lining 424 preferably provides a comfortable contact surface that conducts heat away from the patient's body and also provides an additional frictional force between the pads and the patient's body. The lining 424 can be connected to the pads in any suitable manner, such as with adhesives, elastic bands, snap-fit connections, buckles, clips, rivets, press-fit connections, by integrating the lining with the pads, etc. Also, the lining 424 can include an elastic band connected to its perimeter so that the elastic band can be stretched and wrapped around the perimeter of each pad. One preferred type of material for the lining 424 is a styrene butadiene foam preferably approximately 1 mm to 10 mm thick (e.g., THERMASORB® by Frisby Technologies, Inc.). The lining 424 can also be constructed of one or more other suitable materials (whether capable of directing heat away from the patient's body or not), such as closed-cell foams, soft plastics, or fabrics (e.g., cotton and cotton blends).

In general, the hyperextension orthosis 400 is used to apply pressure to preferably three points on the patient's torso in order to restrict flexion and stabilize the patient's spine. The chest support 10 provides posterior-directed pressure to the patient's chest and the pelvic pad 416 provides posterior-directed pressure to the patient's pelvis. These two posterior-directed pressures are countered with anterior-directed pressure provided by the straps 414 and the posterior pad 415. The hyperextension orthosis 400 is generally suitable for restricting flexion of a patient's torso and for stabilizing a patient's spine, and the hyperextension orthosis 400 is particularly suitable for patients with osteoporosis or compression fractures. Preferably, the components of the hyperextension orthosis 400 are light-weight and have a low-profile design so that the patient can wear the hyperextension orthosis 400 under loose-fitting clothing or over most any apparel. Also, the longitudinal brace member 402 and the lateral brace member 404 preferably do not contact the patient's body (i.e., only the left abdominal pad 406, the right abdominal pad 410, the posterior pad 415, the pelvic pad 416, and the sternal plate 12 contact the patient's body).

As shown in FIGS. 13A and 13B, the chest support 10 can also be used in conjunction with other hyperextension orthoses, such as a Danforth hyperextension orthosis (“DHO”) 500. The DHO 500 includes an abdominal pad 502 connected to a back pad 504 by one or more straps 506. The abdominal pad 502 provides posterior-directed pressure to the patient's abdomen. The abdominal pad 502 is preferably molded to conform to a patient's abdomen. The back pad 504 provides anterior-directed pressure to the patient's back. The back pad 504 is preferably molded to conform to a patient's back. The abdominal pad 502 and the back pad 504 can be constructed of molded plastic, composite, nylon, fiberglass, etc. Preferably, the straps 506 that couple the abdominal pad 502 to the back pad 504 are adjustable.

The chest support 10 is connected to the abdominal pad 502 of the DHO 500 in order to provide posterior-directed pressure at a substantially fixed position with respect to the patient's chest. Preferably, the inferior portion 46 of the sternal bar 14 is connected to the abdominal pad 502. Preferably, T-nuts or other suitable fasteners are positioned and secured through one or more of the holes 60 in the inferior portion 46 of the sternal bar 14 and through holes 508 in the abdominal pad 502 However, the chest support 10 can be connected to the DHO 500 in any suitable manner (e.g., with bolts, screws, buckles, clips, mating pins and apertures, rivets, threaded connections, snap-fit connections, press-fit connections, etc.) or the chest support 10 can be integral with the abdominal pad 502 of the DHO 500. For example, the sternal bar 14 can be integrally constructed of the same material as the abdominal pad 502 and extend upward from the abdominal pad 502 toward the patient's chest.

The chest support 10 can also be used in conjunction with any number of other torso orthoses. For example, the chest support 10 can be used in conjunction with an orthotic vest constructed of a pliant material encompassing relatively rigid pads. Also, the chest support 10 can be used in conjunction with an anterior torso shell secured to the patient's torso, without the use of a posterior torso shell. In general, the chest support 10 can be secured to the patient's torso in any manner that allows the sternal plate 12 to provide posterior-directed pressure to the patient's chest.

The embodiments described above and illustrated in the figures are presented by way of example only and are not intended as a limitation upon the concepts and principles of the present invention. As such, it will be appreciated by one having ordinary skill in the art that various changes in the elements and their configuration and arrangement are possible without departing from the spirit and scope of the present invention as set forth in the appended claims.

We claim:

1. A chest support for use with a torso orthosis securable to a patient, the chest support comprising:
a sternal plate securable in a substantially fixed position with respect to the patient's chest;
a sternal bar; and
a connection between the sternal plate and the sternal bar, the connection including
a housing having
an exterior; and
an interior recess;
the sternal plate coupled to the exterior of the housing;
the sternal bar dimensioned and positioned for movement within the interior recess so that the sternal bar moves with respect to the sternal plate while the sternal plate remains in the substantially fixed position with respect to the patient's chest as the patient's torso flexes.

2. The chest support of claim 1, wherein the sternal plate is pivotably coupled to the exterior of the housing so that the sternal bar pivots with respect to the sternal plate.

3. The chest support of claim 1, wherein the sternal plate is pivotably coupled to the exterior of the housing so that the sternal bar pivots about at least one of a medial/lateral axis and a superior/inferior axis with respect to the sternal plate.

4. The chest support of claim 1, wherein the sternal plate is pivotably coupled to the exterior of the housing by a hinge that includes a rocker stud rigidly coupled to the sternal plate and pivotally coupled to the housing.

5. The chest support of claim 1, wherein the sternal plate is pivotably coupled to the exterior of the housing by a ball and socket joint.

6. The chest support of claim 1, wherein the sternal bar has an effective length, and the effective length of the sternal bar decreases as the patient's torso bends forward with respect to the patient's pelvis and increases as the patient's torso bends backward with respect to the patient's pelvis.

7. The chest support of claim 6, and further comprising a spring coupled to the sternal bar to bias the sternal bar to increase in effective length as the patient's torso bends backward.

8. The chest support of claim 1, wherein the housing is constructed of an acetal polymer having an enhanced lubricity.

9. The chest support of claim 1, wherein the sternal bar includes a first portion coupled to the sternal plate by the connection and positionable substantially parallel to the patient's sternum, a second portion including a bend, and a third portion including an extension connectable to the torso orthosis.

10. The chest support of claim 9, wherein the extension of the third portion includes at least one hole through which at least one fastener is positionable in order to secure the sternal bar to the torso orthosis.

11. The chest support of claim 1, and further comprising a lock positioned to engage the sternal bar in order to adjust the distance that the sternal bar moves with respect to the sternal plate.

12. The chest support of claim 11, wherein the lock is movable between a first position in which the sternal bar cannot move with respect to the sternal plate and a second position in which the sternal bar can move with respect to the sternal plate.

13. The chest support of claim 11, wherein the sternal bar includes a plurality of indentations and the lock includes a set screw selectively positionable within each one of the plurality of indentations.

14. The chest support of claim 1, wherein at least one of the sternal plate and the sternal bar is constructed of aluminum.

15. The chest support of claim 1, wherein the torso orthosis includes one of an orthotic vest, a hyperextension orthosis, a cruciform anterior spinal hyperextension orthosis, a lumbo-sacral orthosis, a thoracal lumbo-sacral orthosis, and a cervical thoracal lumbo-sacral orthosis.

16. A chest support for use with a torso orthosis securable to a patient, the chest support comprising:
a sternal plate securable in a substantially fixed position with respect to the patient's chest;
a sternal bar; and
a connection coupled between the sternal plate and the sternal bar that allows the sternal bar to move longitudinally while the sternal plate remains in the substantially fixed position with respect to the patient's chest when the patient's torso flexes.

17. The chest support of claim 16, wherein the connection allows the sternal bar to slide longitudinally.

18. The chest support of claim 16, wherein the connection allows the sternal bar to pivot with respect to the sternal plate.

19. The chest support of claim 16, wherein the sternal bar has an effective length, and the effective length of the sternal bar decreases as the patient's torso bends forward with respect to the patient's pelvis and increases as the patient's torso bends backward with respect to the patient's pelvis.

20. The chest support of claim 19, and further comprising a spring coupled to the sternal bar to bias the sternal bar to increase in effective length as the patient's torso bends backward.

21. The chest support of claim 16, wherein:
the connection includes a housing having an exterior and an interior recess;
the sternal plate is coupled to the exterior of the housing; and
the sternal bar is dimensioned and positioned for movement within the interior recess so that the sternal bar moves with respect to the sternal plate when the patient's torso flexes.

22. The chest support of claim 21, wherein the housing is constructed of an acetal polymer having an enhanced lubricity.

23. The chest support of claim 21, wherein the sternal plate is pivotably coupled to the exterior of the housing so that the sternal bar pivots with respect to the sternal plate.

24. The chest support of claim 21, wherein the sternal plate is pivotably coupled to the exterior of the housing so that the sternal bar pivots about at least one of a medial/lateral axis and a superior/inferior axis with respect to the sternal plate.

25. The chest support of claim 21, wherein the sternal plate is pivotably coupled to the exterior of the housing by a hinge that includes a rocker stud rigidly coupled to the sternal plate and pivotally coupled to the housing.

26. The chest support of claim 21, wherein the sternal plate is pivotably coupled to the exterior of the housing by a ball and socket joint.

27. The chest support of claim 16, wherein the sternal bar includes a first portion connected to the sternal plate by the connection and positionable substantially parallel to the patient's sternum, a second portion including a bend, and a third portion including an extension connectable to the torso orthosis.

28. The chest support of claim 27, wherein the extension of the third portion includes at least one hole through which at least one fastener is positionable in order to secure the sternal bar to the torso orthosis.

29. The chest support of claim 25, and further comprising a lock positioned to engage the sternal bar in order to adjust the distance that the sternal bar moves longitudinally.

30. The chest support of claim 29, wherein the lock is movable between a first position in which the sternal bar cannot move longitudinally and a second position in which the sternal bar can move longitudinally.

31. The chest support of claim 29, wherein the sternal bar includes a plurality of indentations and the lock includes a set screw selectively positionable within each one of the plurality of indentations.

32. The chest support of claim 16, wherein at least one of the sternal plate and the sternal bar is constructed of aluminum.

33. The chest support of claim 16, wherein the torso orthosis is one of an orthotic vest, a hyperextension orthosis, a cruciform anterior spinal hyperextension orthosis, a lumbo-sacral orthosis, a thoracal lumbo-sacral orthosis, and a cervical thoracal lumbo-sacral orthosis.

34. A chest support for use with a torso orthosis securable to a patient, the chest support comprising:
a sternal plate positionable in a substantially fixed position with respect to the patient's chest;
a sternal bar;
a connection coupled between the sternal plate and the sternal bar that allows the sternal bar to move a distance with respect to the sternal plate while the sternal plate remains in the substantially fixed position with respect to the patient's chest when the patient's torso flexes; and
a lock positioned to engage the sternal bar in order to adjust the distance that the sternal bar moves with respect to the sternal plate.

35. The chest support of claim 34, wherein the lock is movable between a first position in which the sternal bar cannot move with respect to the sternal plate and a second position in which the sternal bar can move with respect to the sternal plate.

36. The chest support of claim 34, wherein the sternal bar includes a plurality of indentations and the lock includes a set screw selectively positionable within each one of the plurality of indentations.

37. The chest support of claim 34, wherein the connection allows the sternal bar to move longitudinally.

38. The chest support of claim 34, wherein the connection allows the sternal bar to pivot with respect to the sternal plate.

39. The chest support of claim 34, wherein the sternal bar has an effective length, and the effective length of the sternal bar decreases as the patient's torso bends forward with respect to the patient's pelvis and increases as the patient's torso bends backward with respect to the patient's pelvis.

40. The chest support of claim 39, and further comprising a spring coupled to the sternal bar to bias the sternal bar to increase in effective length as the patient's torso bends backward.

41. The chest support of claim 34, wherein:
the connection includes a housing having an exterior and an interior recess;
the sternal plate is coupled to the exterior of the housing; and
the sternal bar is dimensioned and positioned for movement within the interior recess so that the sternal bar moves with respect to the sternal plate when the patient's torso flexes.

42. The chest support of claim 41, wherein the housing is constructed of an acetal polymer having an enhanced lubricity.

43. The chest support of claim 41, wherein the sternal plate is pivotably coupled to the exterior of the housing so that the sternal bar pivots with respect to the sternal plate.

44. The chest support of claim 41, wherein the sternal plate is pivotably coupled to the exterior of the housing so that the sternal bar pivots about at least one of a medial/lateral axis and a superior/inferior axis with respect to the sternal plate.

45. The chest support of claim 41, wherein the sternal plate is pivotably coupled to the exterior of the housing by a hinge that includes a rocker stud rigidly coupled to the sternal plate and pivotally coupled to the housing.

46. The chest support of claim 41, wherein the sternal plate is pivotably coupled to the exterior of the housing by a ball and socket joint.

47. The chest support of claim 34, wherein the sternal bar includes a first portion connected to the sternal plate by the connection and positionable substantially parallel to the patient's sternum, a second portion including a bend, and a third portion including an extension connectable to the torso orthosis.

48. The chest support of claim 47, wherein the extension of the third portion includes at least one hole through which at least one fastener is positionable in order to secure the sternal bar to the torso orthosis.

49. The chest support of claim 34, wherein at least one of the sternal plate and the sternal bar is constructed of aluminum.

50. The chest support of claim 34, wherein the torso orthosis includes one of an orthotic vest, a hyperextension orthosis, a cruciform anterior spinal hyperextension orthosis, a lumbo-sacral orthosis, a thoracal lumbo-sacral orthosis, and a cervical thoracal lumbo-sacral orthosis.

51. A method of supporting a patient's torso, the method comprising:
positioning a sternal plate into contact with the patient's chest in a substantially fixed position;
connecting a sternal bar to the sternal plate; and
moving the sternal bar longitudinally as the patient's torso flexes so that the sternal plate remains in the substantially fixed position with respect to the patient's chest.

52. The method of claim 51, and further comprising sliding the sternal bar longitudinally as the patient's torso flexes.

53. The method of claim 51, and further comprising pivoting the sternal bar with respect to the sternal plate as the patient's torso flexes.

54. The method of claim 51, and further comprising pivoting the sternal bar about at least one of a medial/lateral axis and a superior/inferior axis with respect to the sternal plate as the patient's torso flexes.

55. The method of claim 51, and further comprising decreasing an effective length of the sternal bar as the patient's torso bends forward with respect to the patient's pelvis and increasing the effective length of the sternal bar as the patient's torso bends backward with respect to the patient's pelvis.

56. The method of claim 55, and further comprising biasing the sternal bar to increase in effective length as the patient's torso bends backward with respect to the patient's pelvis.

57. The method of claim 51, and further comprising positioning at least a portion of the sternal bar substantially parallel to the patient's sternum.

58. The method of claim 51, and further comprising connecting the sternal bar to an orthotic brace and securing the orthotic brace to the patient's torso.

59. The method of claim 51, and further comprising adjusting a distance that the sternal bar moves longitudinally.

60. The method of claim 51, and further comprising locking the sternal bar so that the sternal bar cannot move longitudinally.
